(19) United States
(12) Patent Application Publication
(10) Pub. No.: US 2002/0146348 A1
(43) Pub. Date: Oct. 10, 2002

(54) Universal pickle liquor acid analyzer

(76) Inventors: Ronald D. Rodabaugh, Lebanon, OH (US); David M. Price, West Chester, OH (US); Gregory A. Bryant, West Chester, OH (US)

Correspondence Address: FROST BROWN TODD LLC, 2200 PNC Center, 201 E. Fifth Street, Cincinnati, OH, 45202-4182, US

(21) Appl. No.: 10/119,339

(22) Filed: Apr. 9, 2002

Related U.S. Application Data
(60) Provisional application No. 60/282,566, filed on Apr. 9, 2001.

(51) Int. Cl. G01N 33/00

(52) U.S. Cl. 422/68.1; 436/73; 436/84; 436/100; 436/116; 436/135; 436/164; 436/149; 436/150; 422/82.01; 422/82.05; 422/82.02; 422/82.12

(57) ABSTRACT

The present invention relates to a modular analyzer system for the analysis of at least two or more chemical components contained in pickle liquor solution. The analyzer modules include a strong acid analysis module, a weak acid analysis module, an oxidizer analysis module, and a metal ion analysis module. These four modules may be used in any combination, including the use of more than one of the same type of analysis module.

This application is based on and claims priority from U.S. Provisional Patent Application Serial No. 60/282,566, Ronald D. Rodabaugh, David M. Price and Gregory A. Bryant, filed on Apr. 9, 2001.

### FIELD OF INVENTION

The present invention relates to an apparatus and process for the analysis of a pickle liquor solution. More particularly, the present invention relates to a modular analyzer system for the analyses of at least two or more chemical components contained in pickle liquor solution.

### BACKGROUND OF THE INVENTION

Pickling is the process of chemically removing oxides and scale from the surface of a metal. The solution in which pickling occurs is known as the pickle liquor. It can be comprised of strong acids, weak acids, oxidizing agents and/or water. In addition, it may contain dissolved metals and/or salts.

The rate of pickling can be affected by, among other things, acid concentrations, temperature, time of metal immersion and dissolved metal concentration. The nature of the metal oxides present, such as those of iron, chromium and nickel, will also influence the rate of pickling.

Since the acid in the pickling operation can be gradually consumed by the removal of the base metal and scale, additional fresh acid is added along with water, while simultaneously removing dissolved metals, to maintain a uniform cleaning operation. In order to accomplish this, the composition of the pickle liquor in the pickling tanks is typically monitored and maintained within relatively certain parameters.

The present invention provides a device which monitors the composition of pickle liquor as it is being used on an on-going basis.

### SUMMARY OF THE INVENTION

The present invention relates to a method and modular apparatus for determining the concentration of constituents in an aqueous solution and in particular aqueous pickle liquor. The composition of pickle liquors may vary, depending on the type of steel, the productivity of the process line, and other factors specific to a given process line. To accommodate this wide variability, the analyzer of the present invention uses a modular concept. Individual modules to measure individual components can be incorporated into an existing platform without substantial modification to the apparatus hardware, including the number of pumps, instrumentation size and electronics. The variations which may be made to the modules include, but are not limited to, different ion-specific electrodes, light sources, measurement of physical phenomena, such as density, and the use of different reagents.

The modular apparatus comprises a dilution means and at least two interconnectable analysis modules for measuring the concentration of the constituents. The modules are selected from the group consisting of strong acid modules, weak acid modules, oxidizer modules, and metal ion modules, and combinations thereof. A drain means connects the dilution means and the interconnectable modules such that a sample entering the apparatus is delivered to each of the interconnectable modules.

The strong acid module is serially connected to, by the drain means, at least two modules selected from the weak acid module, oxidizer module, and metal ion module. The weak acid module, oxidizer module, and metal ion module are connected in parallel to each other by a drain means and the weak acid module, oxidizer module, and metal ion module follow the strong acid analysis module, based on the direction of fluid flow.

The solution may be initially moved through a heat exchanger prior to the it being moved through a de-bubbler and analysis modules (see FIG. 1). Generally, the heat exchanger maintains the solution at temperature of about 85° F. or lower. The de-bubbler is placed in series following the strong acid module and preceding the weak acid module, the oxidizer module, and the metal ion module.

### DESCRIPTION OF THE DRAWINGS

FIG. 1 is a general schematic diagram of the universal pickle liquor analyzer of the present invention.

FIG. 2 is a schematic diagram of the universal pickle liquor analyzer showing specific analysis modules for the detection and quantification of nitric acid, hydrofluoric acid, nitrogen oxides and iron.

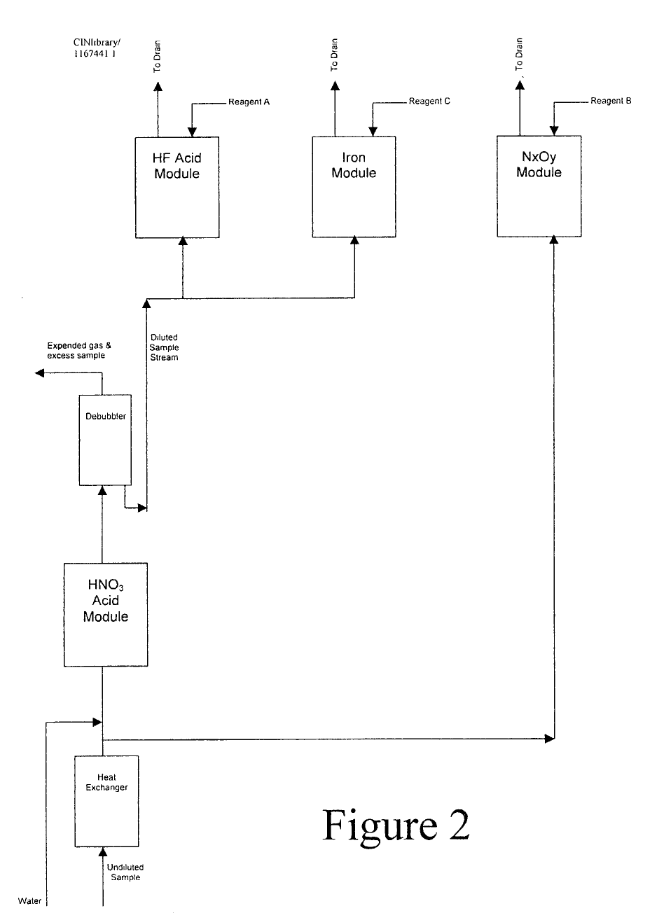

FIG. 3 is a schematic diagram of the universal pickle liquor analyzer showing specific analysis modules for the detection and quantification of sulfuric acid, hydrofluoric acid, hydrogen peroxide and iron.

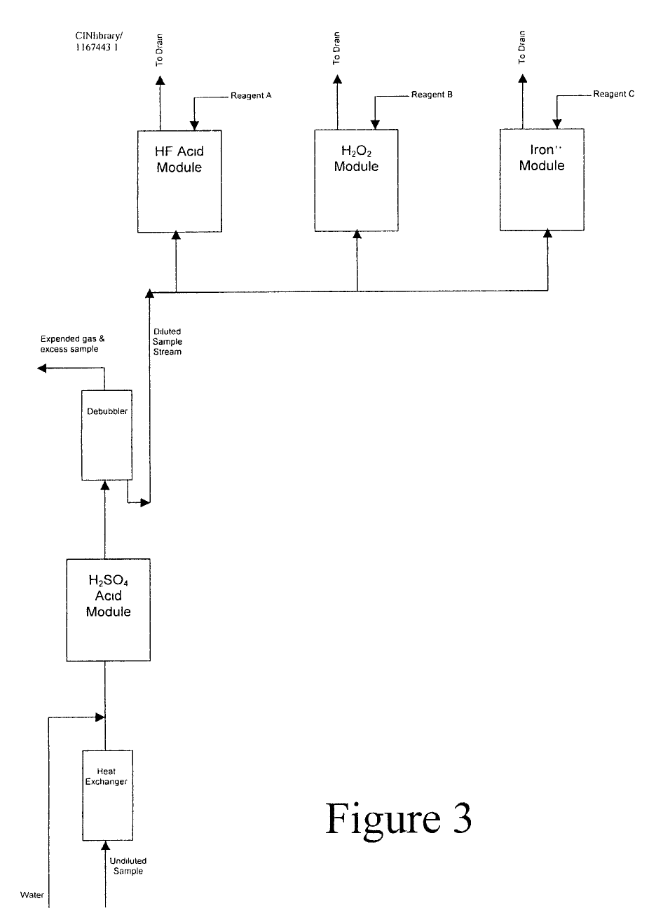

### DETAILED DESCRIPTION OF THE INVENTION

The modular analyzer of the present invention measures the concentration of two or more components of pickle liquor, such that two or more analysis modules will run the majority of their test cycles at the same time, but individual module test cycles may begin or end at different points on the overall analyzer test cycle time line. This is generally accomplished by hooking up at least some of the modules in parallel, rather than series. In a preferred embodiment, the analyses are performed substantially simultaneously. An advantage to this type of analyzer design is that it saves time. Generally, a single sample may be split into separate samples for analysis. The pickle liquor to be analyzed may include strong mineral acids, weak mineral acids, organic acids, dissolved metal ions, such as iron, chromium and nickel, and oxidizing agents such as hydrogen peroxide, potassium permanganate and nitric acid, and combinations thereof.

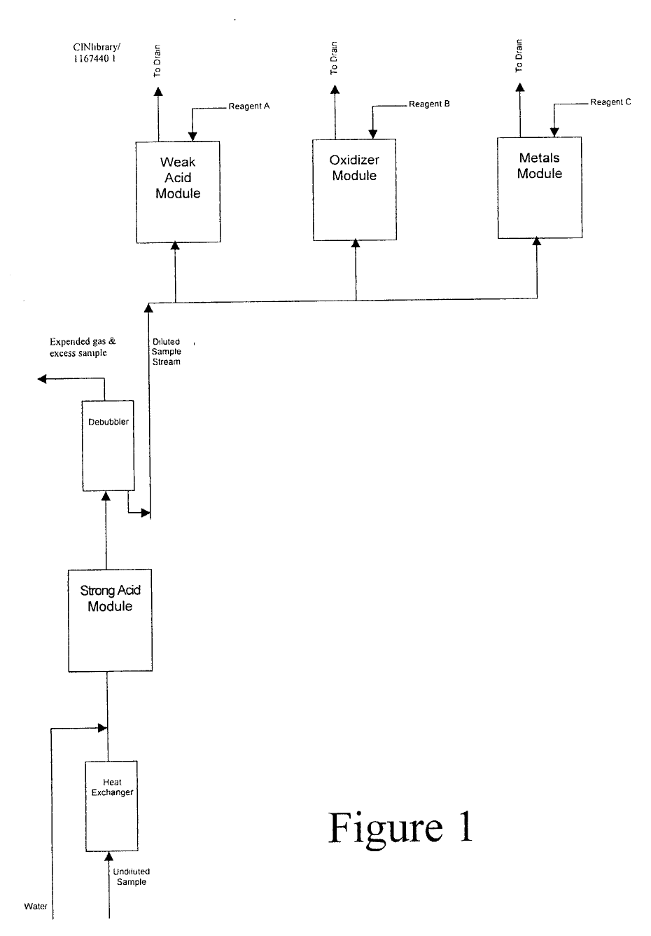

The analyzer modules which may be used in the present invention include a strong acid analysis module, a weak acid analysis module, an oxidizer analysis module, and a dissolved metal ions analysis module. These four modules may be used in any combination of two, three or four or more, including the use of more than one of the same type of analysis module. The strong acid analysis module is generally arranged so that it receives the sample solution first. In a preferred embodiment, the weak acid analysis module, the oxidizer analysis module, and the dissolved metal ion analysis module draw a 2× diluted solution sample from a de-bubbler (see FIG. 1). The de-bubbler allows gas bubbles in the sample to be expelled from one end of the chamber while individual analysis modules extract degassed sample from the opposite end of the chamber, thereby minimizing the interference of gas bubbles on the individual analyses. This is especially important in analysis of samples containing hydrogen peroxide.

All modules are physically connected to the de-bubbler by tubing sections. Solutions may be moved through the modular analyzer using a drain means, which as used herein means a method of physically interconnecting the individual modules so that the same sample solution flows through all modules used in the analyzer apparatus. This may be accomplished by using interconnected tubing and peristaltic pumps.

Strong Acid Analysis Module

When a strong acid is present in the pickle liquor, the strong acid analysis module comprises a conductivity probe for detecting and measuring the presence of strong acids, such as hydrochloric acid, sulfuric acid and nitric acid. The conductivity measurement determines how well the sample conducts alternating current. The conductivity depends upon the concentration and specific conductance of all of the ionic species in the sample. On a per ion basis, the H⁺ from strong acid has about 5× the equivalent conductance of other ions in the sample such as Fe²⁺, NO₃⁻, Cl⁻, or SO₄²⁻. This makes conductivity a good measure of strong acid concentration. The conductivity of other ions cannot be neglected, however. Corrections for the contributions of less strong acid ions, such as thosefrom ionized metal salts, should be applied before calculating strong acid concentration. Conductivity is also temperature dependent and must be corrected to a reference temperature value. Approximate variation with temperature is about 0.8% relative conductivity increase per degree F increase. Pickle liquor solutions are usually diluted with water prior to the conductivity measurement (see FIG. 1). The algorithm used to convert conductivity measurements to acid concentrations is known to those skilled in the art.

When two strong acids are present in the pickle liquor, two separate strong acid modules may be used, wherein one module measures the conductivity of the strong acid solution and the second module may measure for the presence of a specific ion associated with one of the strong acids. For instance, one module would measure the sum of sulfuric and hydrochloric acid concentration by conductivity and the second module measure for the presence of hydrochloric acid concentration by the use of a chloride ion-specific electrode. Manipulation of the values obtained from these modules permits determination of the concentration of each of the acids

Weak Acid Analysis Module

When a weak acid is present in the pickle liquor, the weak acid analysis module detects and measures the concentration of the weak acid by one of two methods. The heat of reaction method measures the temperature rise when the 2× diluted sample is combined with an equal flow of boric acid reagent (1). This technique is specific for hydrofluoric acid in pickle liquors where hydrofluoric acid concentrations are about 10 g/l or higher, since pickle liquors do not contain substantial concentrations of other substances that react with boric acid. However, in some cases, the observed temperature rise is corrected for effects of the heat of dilution caused by further dilution of the 2× diluted sample and the temperature rise is also corrected for the “water blank” which is the observed temperature rise when water is run as the sample.

4HF+H₃BO₃→HBF₄+3H₂O (1)

For hydrofluoric acid concentrations below about 10 g/l, the fluoride ion specific ion electrode method is preferred. In this method, total dilution of the sample is about 56×. This analysis is quite specific for hydrofluoric acid, but is affected by the total proton strength (H⁺ activity) (2). The electrode actually measures free fluoride ion activity. Therefore, strong acid concentration must be measured and the appropriate correction applied to the fluoride ion potential measurement before the hydrofluoric acid concentration is calculated.

HF⇄H⁺+F⁻[F⁻]=[HF]/[H⁺] (2)

Oxidizer Analysis Module

When an oxidizer is present in the pickle liquor, the oxidizer analysis module comprises at least one temperature sensor to measure the heat of reaction of the oxidizer with an appropriate reagent in order to detect and measure the concentration of oxidizer present in solution. In a typical analysis, total dilution of the sample is about 8.75× for the oxidizer module when measuring heat of reaction for hydrogen peroxide. Alternatively, the oxidizer analysis module may also detect and measure the concentration of the oxidizer by redox potential or by differential conductivity measurements with an appropriate reagent, including but not limited to ferrous ion. Examples of oxidizers include, but are not limited to hydrogen peroxide, potassium permanganate, nitrogen oxides, and combinations thereof.

Metal Ion Analysis Module

When dissolved metal ions are present in the pickle liquor, the metal ion analysis module comprises a photometric cell for photometrically detecting and measuring the concentration of metal ions present in the pickle liquor. In a typical analysis, total dilution of the sample is about 29× for the metal ion module. The metal ions are detected by the reaction of the metal ion with an appropriate ligand for photometric detection. Examples of metal ions include, but are not limited to iron, nickel and chromium. The resulting metal-ligand complex may absorb in the ultraviolet, near ultraviolet, visible or near infrared region of the electromagnetic spectrum. Suitable ligands include, but are not limited to, citrate, ortho-phenanthroline and thiocyanate. Alternatively, the metal ion analysis module may measure sample density. Metal ion concentration can then be calculated from the density, once the density has been corrected for the effects of acids in the sample. Also, alternatively, metal ion concentration can be determined from differential conductivity measurements after adding an appropriate reagent to the sample.

Discussion

The modules of the analyzer of the present invention can be exchanged in order to perform the particular measurements of interest, depending upon the constituents in the pickle liquor. For example, a temperature-sensing module used to measure hydrogen peroxide concentration by heat of reaction with ferrous iron could be replaced with an ion specific electrode module to measure chloride ion concentration. In addition, the modules allow for the simplest chemistry to be used with each component measurement. Conversely, if samples were passed sequentially through several modules, reagents introduced in earlier modules could interfere with measurements performed in later modules. The parallel analysis design prevents cross-contamination of the samples prior to analysis.

In the apparatus of the present invention, a sample containing at least one constituent is passed through a heat exchanger in order to maintain the sample solution at a temperature of about 85° F. or lower. The sample may then be diluted with water. The initial dilution of the sample with water is about a 2× dilution. Conductivity is measured on this diluted sample. Since conductivity is a physical property measurement, the chemical characteristics of the 2× diluted sample stream are not altered by the conductivity measurement. When a sample is tested for a single strong acid concentration, the analysis is, with respect to the strong acid analysis module, sequential with respect to the other remaining analysis modules (see for example, FIG. 1).

Once the sample has flowed through the strong acid analysis module, the diluted sample is substantially simultaneously delivered to two or more testing modules. All modules, with the exception of the conductivity module, which uses the 2× dilution sample, and the nitrogen oxides module, which uses undiluted sample, draw upon the sample stream from the de-bubbler for analysis.

All modules are interfaced with a computer which performs the necessary calculations and reports the results of the analyses.

Calibration:

Calibration against standard solutions should be performed on a periodic basis. These calibration tests can include conductivity, differential temperature change, specific ion electrode response and photometric response.

### EXAMPLES

### Example 1

### H₂SO₄/HF/Fe³⁺/H₂O₂

The pickle liquor sample is moved through a heat exchanger and cooled to about 75° F. The sample is then diluted with an equal volume of water. The sample is moved through the strong acid module, where conductivity and temperature measurements are taken. The conductivity value is corrected for temperature and interactions from hydrofluoric acid. The corrected value is then used to calculate sulfuric acid concentration.

Substantially simultaneously, a portion of the diluted sample is moved through the hydrofluoric acid (weak acid) module. Inside this module, the sample is combined with a boric acid reagent. The temperature of the incoming sample (T1) and the temperature of the boric acid reagent (T2) are individually measured. The temperature of the resulting mixture is also measured (T3). If the flow of the sample and reagent streams are equal, the hydrofluoric acid concentration calculation has the form of:

HF concentration=empirical factor×[T3−(½×T1)−(½×T2)−water blank]

Alternatively, the HF measurement module may consist of an ion-specific electrode. Inside the module, the sample is further diluted with additional water. The fluoride potential is measured, and the HF concentration calculated from the fluoride potential, after the fluoride potential has been corrected for the effect of H₂SO₄ concentration.

A portion of the diluted sample is substantially simultaneously moved through the hydrogen peroxide module. Inside this module, the sample is combined with a ferrous iron reagent. The temperature of the incoming sample (T1) and the temperature of the ferrous iron reagent (T2) are individually measured. The temperature of the resulting mixture is also measured (T3). The sample stream flow rate is about 8 ml/min, and reagent stream flow rate is about 27 ml/min. The H₂O₂ concentration calculation has the form of:

H₂O₂ concentration=empirical factor×[T3−(8/35×T1)−(27/35×T2)−water blank]

A portion of the diluted sample is substantially simultaneously moved through the iron (ferric and/or ferrous ions) module. Inside the module, the sample is combined with the buffered citric acid reagent also containing boric acid and H₂O₂. A photometric method is used to measure light absorption of the yellow ferric-citrate complex. The light absorption of the complex is measured. The light absorption of water is measured. The iron concentration is then calculated from the ratio of the sample complex absorption as compared to the water absorption.

### Example 2

### HCl/HF/Fe²⁺

The pickle liquor sample is moved through a heat exchanger and cooled to about 75° F. The sample is then diluted with an equal volume of water.

The sample is moved through the strong acid module where the conductivity and temperature are measured. The conductivity value is corrected for temperature and interactions from hydrofluoric acid and ferrous iron. The corrected value is then used to calculate hydrochloric acid concentration.

portion of the diluted sample is substantially simultaneously moved through the hydrofluoric acid (weak acid) module. Inside the module, the sample is combined with a boric acid reagent. The temperature of the incoming sample (T1) and the temperature of the boric acid reagent (T2) are individually measured. The temperature of the resulting mixture is also measured (T3). If the flow of the sample and reagent streams are equal, the HF concentration calculation has the form of:

HF concentration=empirical factor×[T3−(½×T1)−(½×T2)−water blank].

Alternatively, the HF measurement module may consist of an ion-specific electrode. Inside the module, the sample is further diluted with additional water. The fluoride potential is measured, and the HF concentration calculated form the fluoride potential, after the fluoride potential has been corrected for the effect of HCl concentration.

A portion of the diluted sample is substantially simultaneously moved through the iron (ferric and/or ferrous ions) module. Inside this module, the sample is combined with the buffered citric acid reagent also containing boric acid and H₂O₂. A photometric method is used to measure light absorption of the yellow ferric-citrate complex. The light absorption of the complex is measured. The light absorption of water is measured. The iron concentration is then calculated from the ratio of the sample complex absorption as compared to the water absorption.

### Example 3

### HNO₃/HF/nitrogen oxides/Fe⁺³

The pickle liquor sample is moved through a heat exchanger and cooled to about 75° F.

A portion of the undiluted sample is moved through the NₓO_{y} module. Inside the module, the sample is combined with a sulfamic acid reagent. The temperature of the incoming sample (T1) and the temperature of the sulfamic acid reagent (T2) are individually measured. The temperature of the mixture is also measured (T3). The sample stream flow rate is about 27 ml/min, and reagent stream flow rate is about 8 ml/min. The NₓO_{y} concentration calculation has the form of:

NₓO_{y} concentration=empirical factor×[T3−(27/35×T1)−(8/35×T2)−water blank]

The sample is then diluted with an equal volume of water. The sample is moved substantially simultaneously through the strong acid module where the conductivity and temperature are measured. The conductivity value is corrected for temperature and interactions from hydrofluoric acid. The corrected value is then used to calculate nitric acid concentration.

A portion of the diluted sample is substantially simultaneously moved through the hydrofluoric acid module. Inside this module, the sample is combined with a boric acid reagent. The temperature of the incoming sample (T1) and the temperature of the boric acid reagent (T2) are individually measured. The temperature of the resulting mixture is also measured (T3). If the flow of the sample and reagent streams are equal, the HF concentration calculation has the form of:

HF concentration=empirical factor×[T3−(½×T1)−(½×T2)−water blank].

Alternatively, the HF measurement module may consist of an ion-specific electrode. Inside the module, the sample is further diluted with additional water. The fluoride potential is measured, and the HF concentration calculated form the fluoride potential, after the fluoride potential has been corrected for the effect of HNO₃ concentration.

A portion of the diluted sample is substantially simultaneously moved through the iron (ferric and/or ferrous ions) module. Inside this module, the sample is combined with the buffered citric acid reagent also containing boric acid and H₂O₂. A photometric method is used to measure light absorption of the yellow ferric-citrate complex. The light absorption of the complex is measured. The light absorption of water is measured. The iron concentration is then calculated from the ratio of the sample complex absorption as compared to the water absorption.

### Example 4

### H₂SO₄/HCl/Fe²⁺

The pickle liquor sample is moved through a heat exchanger and cooled to about 75° F. The sample is then diluted with an equal volume of water.

The sample is moved through the strong acid module where the conductivity and temperature are measured. The conductivity value is corrected for temperature and interactions from hydrochloric acid and ferrous iron. The corrected value is then used to calculate sulfuric acid concentration.

The hydrochloric acid measurement module consists of an ion-specific electrode. Inside the module, the sample is further diluted with water. The chloride potential is measured, and the HCl concentration calculated from the chloride potential.

A portion of the diluted sample is substantially simultaneously moved through the iron (ferric and/or ferrous ions) module. Inside this module, the sample is combined with the buffered citric acid reagent also containing boric acid and H₂O₂. A photometric method is used to measure light absorption of the yellow ferric-citrate complex. The light absorption of the complex is measured. The light absorption of water is measured. The iron concentration is then calculated from the ration of the sample complex absorption as compared to the water absorption.

What is claimed is:

1) A modular analyzer apparatus for determining the concentration of constituents in an aqueous solution, said analyzer apparatus comprising:
a) a solution dilution means;
b) at least two interconnectable analysis modules for measuring and reporting the concentration of said constituents, wherein said modules are selected from the group consisting of strong acid modules, weak acid modules, oxidizer modules, and metal ion modules, and combinations thereof; and
c) drain means connecting said solution dilution means and said at least two interconnectable modules such that a sample entering the apparatus is delivered to each of said at least two interconnectable modules.

2) The analyzer of claim 1 wherein the strong acid module is serially connected to, by the drain means, at least two modules selected from the weak acid module, oxidizer module, and metal ion module.

3) The analyzer of claim 2 wherein at least two modules selected from the weak acid module, oxidizer module, and metal ion module are connected in parallel to each other by drain means and the weak acid module, oxidizer module, and metal ion module follow the strong acid analysis module, based on the direction of fluid flow.

4) The analyzer of claim 3 wherein the aqueous solution to be analyzed comprises pickle liquor.

5) The analyzer of claim 4 wherein the strong acid module measures the concentration of strong acids by conductivity.

6) The analyzer of claim 5 wherein the strong acids are selected from the group consisting of hydrochloric acid, nitric acid and sulfuric acid.

7) The analyzer of claim 4 wherein the strong acid module measures the concentration of strong acids by measuring for the presence of a specific ion associated with the strong acid.

8) The analyzer of claim 4 wherein the weak acid module measures and reports the concentration of weak acids by using an ion-specific electrode for measurement of an anion associated with the weak acid.

9) The analyzer of claim 4 wherein the weak acid module measures and reports the concentration of weak acids by using at least one temperature sensor to measure a heat of reaction of the weak acid with an appropriate reagent.

10) The analyzer of claim 4 wherein the oxidizer module measures the concentration of an oxidizer, and the oxidizer is selected from the group consisting of hydrogen peroxide, potassium permanganate, nitrogen oxides, and combinations thereof.

11) The analyzer of claim 10 wherein the concentration of the oxidizer is measured by at least one temperature sensor to measure the heat of reaction of the oxidizer with an appropriate reagent.

12) The analyzer of claim 10 wherein the concentration of the oxidizer is measured by differential conductivity measurements with an appropriate reagent.

13) The analyzer of claim 4 wherein the metal ion analysis module measures the concentration of metal ions photometrically.

14) The analyzer of claim 13 wherein the metal ions are reacted with a ligand to form a metal-ligand complex for photometric detection.

15) The analyzer of claim 14 wherein the metal-ligand complex absorbs light in the near ultraviolet, ultraviolet, visible or near infrared region of the electromagnetic spectrum.

16)The analyzer of claim 15 wherein the metal ion is selected from the group consisting of iron, chromium and nickel ions.

17) The analyzer of claim 16 wherein the ligand is selected from the group consisting of citrate, ortho-phenanthroline and thiocyanate.

18) The analyzer of claim 4 wherein the solution is initially moved through a heat exchanger prior to the solution being moved through the de-bubbler and analysis modules.

19) The analyzer of claim 18 wherein the heat exchanger maintains the solution at temperature of about 85° F. or lower.

20) The analyzer of claim 19 wherein a de-bubbler is placed in series following the strong acid module and preceding the weak acid module, the oxidizer module, and the metal ion module.

21) A method for analyzing pickle liquor solution, said method comprising the steps of:
a) providing the pickle liquor solution, which is comprised of strong acids, weak acids, oxidizers, metal ions and combinations thereof;
b) moving said solution through a heat exchanger to maintain the sample solution at temperature of about 85° F. or lower;
c) diluting said solution with an equal volume of water;
d) moving said solution through a strong acid module for determination of strong acid concentration;
e) moving said solution through a de-bubbler which is arranged serially with the modules;
f) moving said solution through at least two additional interconnectable analysis modules, wherein said modules are connected serially with said strong acid module and in parallel with each other by the drain means, said additional modules selected from the group consisting of a weak acid module, an oxidizer module, and a metal ion module and combinations thereof, and wherein each of said modules measures and reports the concentration of a single constituent.

22) The method of claim 21 wherein the strong acid module measures the concentration of strong acids by conductivity.

23) The method of claim 22 wherein the weak acid module measures the concentration of weak acids by using an ion specific electrode for measurement of an anion associated with the weak acid.

24) The method of claim 23 wherein the oxidizer module comprises at least one temperature sensor to measure the heat of reaction of the oxidizer with an appropriate reagent.

25) The method of claim 24 wherein the metal ion analysis module measures the concentration of metal ions photometrically by reacting a metal ion with a ligand to form a metal-ligand complex for photometric detection.
